(19) United States
(12) Patent Application Publication
(10) Pub. No.: US 2010/0256596 A1
(43) Pub. Date: Oct. 7, 2010

(54) FIBER GROWTH PROMOTING IMPLANTS FOR REDUCING THE APPEARANCE OF CELLULITE

(75) Inventor: James E. Chomas, San Carlos, CA (US)

Correspondence Address: FULWIDER PATTON LLP, 6060 CENTER DRIVE, TENTH FLOOR, LOS ANGELES, CA, 90045, US

(73) Assignee: CABOCHON AESTHETICS, INC., Menlo Park, CA (US)

(21) Appl. No.: 12/419,905

(22) Filed: Apr. 7, 2009

(51) Int. Cl. A61M 5/31; A61M 5/20; A61F 2/02

(52) U.S. Cl. 604/506; 604/62; 623/23.72

(57) ABSTRACT

A dermatological skin treatment device is provided. The device comprises implant injection device, comprising, a control, an implant loader, an implant ejection port, and inserts at least one implant into a treatment area, the implant comprising a biocompatible material having a nominal length from perpendicular about 5 mm to 20 mm, the length being substantially longer than a nominal outer diameter of the implant, wherein the implant has at least two barbs preferably configured in an opposing pattern and mirrored along each respective half of the implant. The device and method inserts the implants under the skin to form new fibrous structures in a subdermal treatment area to counteract cellulite by creating a highly fibrous layer directly or through wound healing processes.

### FIELD OF THE INVENTION

The present invention relates to implantable devices which decrease the appearance of cellulite by increasing collagen thickness and to devices and methods for implanting the same.

### BACKGROUND

Gynoid lipodystrophy is a localized metabolic disorder of the subcutaneous tissue which leads to an alteration in the topography of the cutaneous surface (skin), or a dimpling effect caused by increased fluid retention and/or proliferation of adipose tissue in certain subdermal regions. This condition, commonly known as cellulite, affects over 90% of post-pubescent women, and some men. Cellulite commonly appears on the hips, buttocks and legs, but is not necessarily caused by being overweight, as is a common perception. Cellulite is formed in the subcutaneous level of tissue, in the subdermal fat layer below the epidermis and dermis layers. In this region, fat cells are arranged in chambers surrounded by bands of connective tissue called septae. Cellulite is in part due to the parallel orientation of these fibrous septae structures. The fibrous structures being oriented in a parallel fashion (and perpendicular to the skin) is unique to women, whereas men typically have more random orientation of fibrous structures. This difference in fibrous structure may be in part or wholly responsible for the fact that men do not exhibit widespread cellulite in comparison to women. As water is retained, fat cells held within the perimeters defined by these fibrous septae expand and stretch the septae and surrounding connective tissue. Furthermore, adipocyte expansion from weight gain may also stretch the septae. Eventually this connective tissue contracts and hardens (scleroses) holding the skin at a non-flexible length, while the chambers between the septae continue to expand with weight gain, or water gain. This results in areas of the skin being held down while other sections bulge outward, resulting in the lumpy, ‘orange peel’ or ‘cottage cheese’ appearance on the skin surface. Even though obesity is not considered to be a root cause of cellulite, it can certainly worsen the dimpled appearance of a cellulitic region due to the increased number of fat cells in the region.

A variety of approaches for treatment of skin irregularities such as cellulite and removal of unwanted adipose tissue have been proposed. For example, methods and devices that provide mechanical massage to the affected area, through either a combination of suction and massage or suction, massage and application of energy, in addition to application of various topical agents are currently available. Developed in the 1950's, mesotherapy is the injection of various treatment solutions through the skin that has been widely used in Europe for conditions ranging from sports injuries to chronic pain, to cosmetic procedures to treat wrinkles and cellulite. The treatment consists of the injection or transfer of various agents through the skin to provide increased circulation and the potential for fat oxidation, such as aminophylline, hyaluronic acid, novocaine, plant extracts and other vitamins. The treatment entitled Acthyderm (Turnwood International, Ontario, Canada) employs a roller system that electroporates the stratum corneum to open small channels in the dermis, followed by the application of various mesotherapy agents, such as vitamins, antifibrotics, lypolitics, anti-inflammatories and the like.

Massage techniques that improve lymphatic drainage were tried as early as the 1930's. Mechanical massage devices, or Pressotherapy, have also been developed such as the “Endermologie” device (LPG Systems, France), the “Synergie” device (Dynatronics, Salt Lake City, Utah) and the “Silklight” device (Lumenis, Tel Aviv, Israel), all utilizing subdermal massage via vacuum and mechanical rollers. Other approaches have included a variety of energy sources, such as Cynosure's “TriActive” device (Cynosure, Westford, Mass.) utilizing a pulsed semiconductor laser in addition to mechanical massage, and the “Cellulux” device (Palomar Medical, Burlington, Mass.) which emits infrared light through a cooled chiller to target subcutaneous adipose tissue. The “VelaSmooth” system (Syneron, Inc., Yokneam Illit, Israel) employs bipolar radiofrequency energy in conjunction with suction to increase metabolism in adipose tissue, and the “Thermacool” device (Thermage, Inc., Hayward, Calif.) utilizes radiofrequency energy to shrink the subdermal fibrous septae to treat wrinkles and other skin defects. Other energy-based therapies such as electrolipophoresis, using several pairs of needles to apply a low frequency interstitial electromagnetic field to aid circulatory drainage have also been developed. Similarly, non-invasive ultrasound is used in the “Dermosonic” device (Symedex Medical, Minneapolis, Minn.) to promote reabsorption and drainage of retained fluids and toxins.

Methods and devices using ultrasound to disrupt subcutaneous tissues directly have been described in the known art. Such techniques may utilize a high intensity ultrasound wave that is focused on a tissue within the body, thereby causing a localized destruction or injury to cells. The focusing of the high intensity ultrasound may be achieved utilizing, for example, a concave transducer or am acoustic lens. Use of high intensity focused ultrasound to disrupt fat, sometimes in combination with removal of the fat by liposuction, has been described in the known prior art. Such use of high intensity focused ultrasound should be distinguished from the low acoustic pressure ultrasound.

The “Lysonix” system (Mentor Corporation, Santa Barbara, Calif.) utilizes an ultrasonic transducer on the handpiece of the suction cannula to assist in tissue disruption (by cavitation of the tissue at the targeted site). Liposonix (Bothell, Wash.) and Ultrashape (Tel Aviv, Israel) employ the use of focused ultrasound to destroy adipose tissue noninvasively. In addition, cryogenic cooling has been proposed for destroying adipose tissue. A variation on the traditional liposuction technique known as tumescent liposuction was introduced in 1985 and is currently considered by some to be the standard of care in the United States. It involves the infusion of tumescent fluids to the targeted region prior to mechanical disruption and removal by the suction cannula. The fluids may help to ease the pain of the mechanical disruption, while also swelling the tissues making them more susceptible to mechanical removal. Various combinations of fluids may be employed in the tumescent solution including a local anesthetic such as lidocaine, a vasoconstrictive agent such as epinephrine, saline, potassium and the like. The benefits of such an approach are detailed in the articles, “Laboratory and Histopathologic Comparative Study of Internal Ultrasound-Assisted Lipoplasty and Tumescent Lipoplasty” Plastic and Reconstructive Surgery, Sep. 15, 2002 110:4, 11581164, and “When One Liter Does Not Equal 1000 Milliliters: Implications for the Tumescent Technique” Dermatol. Surg. (2000) 26:1024-1028, the contents of which are expressly incorporated herein by reference in their entirety.

Recently, it is has also become possible to exploit ultrasound waves for the purpose of disrupting tissue and tissue ablation without heating tissue to a level of tissue disruption. One such device is disclosed in U.S. Publication No. 2007/0055179 to Deem et al., incorporated herein by reference, which includes a method of infiltrating exogenous microbubbles into the target tissue, and then applying low acoustic pressure ultrasound to the infiltrated tissue to be treated to cavitate the bubbles and destroy the target tissue without direct thermal injury to the tissue. Although low acoustic pressure ultrasound may somewhat heat the tissue, the tissue is not heated sufficiently to cause direct tissue disruption or to enhance the ablation, and thus significantly reduces the risk of thermal damage to the dermis and associated structures (nerves, hair follicles, blood vessels).

Various other approaches employing dermatologic creams, lotions, vitamins and herbal supplements have also been proposed to treat cellulite. Private spas and salons offer cellulite massage treatments that include body scrubs, pressure point massage, essential oils, and herbal products using extracts from plant species such as seaweed, horsetail and clematis and ivy have also been proposed. Although a multitude of therapies exist, most of them do not provide a lasting effect on the skin irregularity and for some, one therapy may cause the worsening of another (as in the case of liposuction causing scarring or a more pronounced appearance of cellulite). Yet other treatments for cellulite have negative side effects that limit their adoption. Most therapies require multiple treatments on an ongoing basis to maintain their effect at significant expense and with mixed results.

Certain other techniques known as liposuction, tumescent liposuction, lypolosis and the like, target adipose tissue in the subdermal and deep fat regions of the body. These techniques may include also removing the fat cells once they are disrupted, or leaving them to be resorbed by the body's immune/lymphatic system. Traditional liposuction includes the use of a surgical cannula placed at the site of the fat to be removed, and then the use of an infusion of fluids and mechanical motion of the cannula to break up the fatty tissue, and suction to “vacuum” the disrupted fatty tissue directly out of the patient. Traditional fat extraction techniques such as liposuction that target deep fat and larger regions of the anatomy, can sometimes worsen the appearance of cellulite since the subdermal fat pockets remain and are accentuated by the loss of underlying bulk (deep fat) in the region. Many times liposuction is performed and patients still seek therapy for remaining skin irregularities, such as cellulite.

Another approach to the treatment of skin irregularities such as scarring and dimpling is a technique called subcision. This technique involves the insertion of a relatively large gauge needle subdermally in the region of dimpling or scarring, and then mechanically manipulating the needle below the skin to break up the fibrous septae in the subdermal region. In at least one known method of subcision, a local anesthetic is injected into the targeted region, and an 18-gauge needle is inserted 10-20 mm below the cutaneous surface. The needle is then directed parallel to the epidermis to create a dissection plane beneath the skin to essentially tear through, or “free up” the tightened septae causing the dimpling or scarring. Pressure is then applied to control bleeding acutely, and then by the use of compressive clothing following the procedure. While clinically effective in some patients, pain, bruising, bleeding and scarring can result.

Destroying fibrous septae in the subdermal region by subcision, however, cannot reprogram a person's genetics or change the genetic orientation of fibrous septae structures. After subcision is performed, it is believed that when the tissue heals the fibrous septae will re-grow in its original orientation and where the structures were originally oriented in a parallel fashion (and perpendicular to the skin), the cellulite will eventually return. As water is retained, or adipocyte is expanded from weight gain, the fat cells held within the perimeters defined by these fibrous septae will expand and stretch the renewed septae and surrounding connective tissue. Eventually this connective tissue will again contract and harden, holding the skin at a non-flexible length, while the chambers between the septae continue to expand, resulting in areas of the skin being held down while other sections bulge outward. For these reasons, and because of the side effects and extended time required for healing, subcision has largely been abandoned as a treatment for cellulite in the United States.

In light of the foregoing, it would be desirable to provide methods and apparatus for treating skin irregularities such as cellulite and to provide a sustained aesthetic result to a body region, such as the face, neck, arms, legs, thighs, buttocks, breasts, stomach and other targeted regions which are minimally or non-invasive. It would also be desirable to provide methods and apparatus for treating skin irregularities that enhance prior techniques and make them less invasive and subject to fewer side effects. The present invention adds fibrous structure scaffolding in the subdermal fat layer, or in the layer between the subdermal fat layers and the skin, at non-parallel angles through the use of scaffolds or through wound healing processes. This treatment may be used in conjunction with known methods of removing fat, skin tightening, or dermal thickening.

### SUMMARY OF THE INVENTION

Disclosed is an implantable device for decreasing the appearance of cellulite by increasing collagen thickness and an implant injection device for implanting the same, comprising a control, an implant actuator, an implant loader, wherein the loader holds at least one implant, ejection port, and a positioning guide disposed between the implant actuator and the ejection port passing adjacent to the feed magazine, wherein the implants comprise a biocompatible material having a nominal length from about 5 mm to 20 mm, the length being substantially longer than a nominal outer diameter of the implant. The device is configured to inject at least one implant into a treatment area comprising the subdermal fat layer, or the layer between the subdermal fat layers and the skin, when the control is actuated to create a new fibrous structure in the treatment area to reduce the appearance of the cellulite.

The implant may have at least two barbs preferably configured in an opposing pattern and mirrored along each respective half of the implant. The implant actuator may also be configured to push the implant percutaneously into the treatment area. In some embodiments the implant injection device further comprises an introducing needle, wherein the introducing needle is configured to receive an implant into a recessed portion of the introducing needle, and to percutaneously insert the implant into the treatment area. In other embodiments the implant injection device further comprises an introducing needle, wherein the introducing needle is configured to thread the implant as it is forwarded to the introducing needle by the implant loader, and to push the implant percutaneously into the treatment area.

The control of the implant injection device may manipulate the depth of the needle, the speed of deployment, or angle of penetration, and the device may be electrically powered. The implant injection device may also comprise a thermal energy device associated with the tissue apposition surface and configured to apply a thermal energy to the treatment area.

Also disclosed is a method of injecting surgical implants for treating cellulite, comprising providing an implant injection device, comprising, a control, an implant loader, an implant ejection port, and preparing a treatment area comprising a section of skin, and inserting an implant into the implant loader, the implant comprising a biocompatible material having a nominal length from about 5 mm to 20 mm. The length of the implant is substantially longer than a nominal outer diameter of the implant, wherein the implant has at least two barbs preferably configured in an opposing pattern and mirrored along each respective half of the implant. The method further comprises positioning the implant ejection port proximal the treatment area, and actuating the control to inject at least one implant into the treatment area to create a new fibrous structure in a subdermal tissue to reduce the appearance of the cellulite.

In this method, the implant injection device is moved substantially parallel to the treatment area while the control is actuated, and wherein at least two implants are fired into the treatment area in at least two locations. The control may also be manipulated to set a speed or depth of deployment, or apply a thermal energy to the treatment area. The treatment area may be prepared by stretching at least a portion of the skin, and injecting the implant will support at least a portion of the skin in a new position.

The disclosed implant of the present invention preferably comprises a biocompatible material having a nominal length from about 5 mm to 20 mm, the length being substantially longer than a nominal outer diameter of the implant, wherein the implant has at least two barbs preferably configured in an opposing pattern and mirrored along each respective half of the implant.

The biocompatible material may have a nominal outer diameter from about 0.25 mm to 0.50 mm, and may be rigid and/or pliable. In one embodiment a cross-section of the implant is substantially flat, while in another embodiment a cross-section of the implant is substantially round. In one embodiment the biocompatible material is bioabsorbable. The biocompatible material may also be a resiliently expandable elastomeric material that can recover its reduced size after insertion into the body. The biocompatible material of the implant may further provide fluid permeability through the material and permits cellular in growth and proliferation into the interior of the material such as to allow it to bind into the surrounding tissues. The biocompatible material may also be reticulated, such that it is comprised of an interconnected network of pores, either by being formed having a reticulated structure and/or undergoing a reticulation process.

### BRIEF DESCRIPTION OF THE DRAWINGS

FIG. 1 depicts the implant of the present invention.

FIG. 2A depicts an implementation of a treatment area that is modified with implants.

FIG. 2B depicts newly formed fibrous structure scaffolding in the subdermal fat layer after the wound healing process.

FIGS. 3A-3E depicts the implant injection device of the present invention and operation thereof.

### DETAILED DESCRIPTION OF THE PREFERRED EMBODIMENTS

Cellulite is in part due to the parallel orientation of fibrous structures in the subdermal fat layer. The fibrous structures being oriented in a parallel fashion (and perpindicular to the skin) is unique to women; moreover, men typically have more random orientation of fibrous structures and this different in fibrous structure may be in part or wholly responsible for the fat that men do not exhibit widespread cellulite in comparison to women. In general, the invention utilizes a device and method to rapidly and minimally-invasively inject sutures, quills, fibers or fiber promoting material such as proteins, actin, collagen, etc., into the subdermal fat layer, or in the layer between the subdermal fat layers and the skin, at a sufficient angle to counteract the predominantly parallel structures of the fibrous septae in women.

The procedure of the present invention involves pulling the skin so as to create a shear effect between the top skin and the underlying fat. This will also pull the skin closer to the underlying structures. Then while maintaining the shear effect, one or many sutures, quills, fibers or fiber promoting molecules are injected and then the skin is released. The procedure is repeated several times with the skin pulled in a different direction each time. This could be performed on a set of different directions with the goal being to make the sutures substantially non-parallel in their orientation.

Throughout this disclosure the term implant will be used to refer generally to any foreign body which is implanted into subcutaneous tissue, and which may bind into the surrounding tissue. The implant may refer to sutures, filaments, fibers, fibrous structures, scaffolding, quills or the like. The implants used in any of the embodiment described herein may be bioabsorbable such that the implant dissolves or is otherwise absorbed by the body over time. The implants may be heat sensitive and shrink when they are heated. Each of the embodiments disclosed herein may be used to treat targeted areas affected by cellulite, for example, the upper leg below the buttocks where cellulite is most visible. Upon implantation, the implants reduce dimpling by the nature of their structural properties. Over long term, e.g., 3-6 months after implantation, the implants promote more fibrous tissue which further reduces the appearance of cellulite.

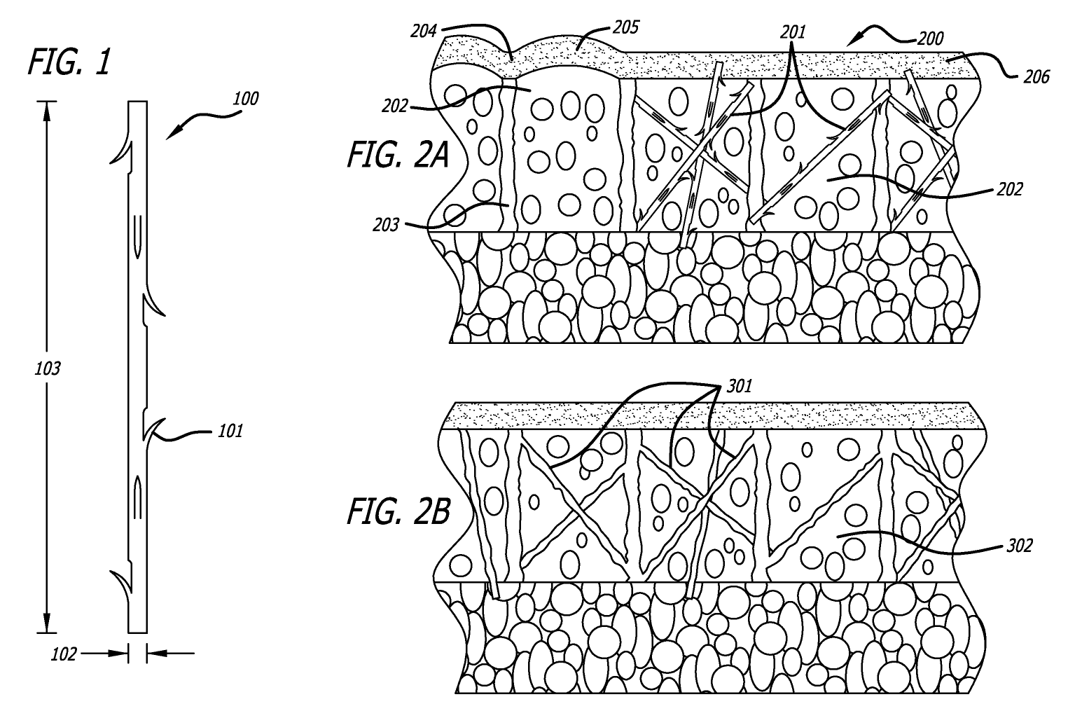

FIG. 1 depicts an implant 100 of the present invention. The implants of the present invention are small surgical threads, quills, or sutures that comprise tiny protruding barbs to help them adhere to tissue underneath the skin. Implant 100 preferably is longer than it is wide and has multiple barbs 101 along a length 103 of the implant. The barbs are preferably configured in an opposing pattern mirrored along each respective half of the implant, or at or near its ends. Barbs 101 also are preferably sized and sufficiently firm such as to maintain the position of the implant and secure the skin when placed under considerable stress, without the need for additional suturing on the exterior of the body. The implants are also preferably fashioned suitably narrow and short in length such that they can be individually placed into or under the skin in a manner that, after several days of healing, their detection would go relatively unnoticed. It is the intent of the invention that the implant may be percutaneously injected in what is touted as a “scarless” cellulite reduction procedure.

Generally, the implant preferably maintains a narrow profile such that it can be percutaneously inserted with minimal invasion to the treatment area. The nominal outer diameter 102 of the implant typically ranges from 0.25 mm to 0.50 mm but can be smaller or larger depending on the skin type and/or tolerance of the patient. Generally, each implant has a nominal length 103 from about 5 mm to 20 mm, however, the implant can have a smaller or larger length depending on several factors, including the area to be treated and/or skin type. For the purposes of illustration, the implant has a substantially flat cross-section. Other embodiments may include implants that are curved, bowed, or angled, rectangular, triangular, square, round, or any other design which could be useful in improving the strength of the implants and/or effectiveness of treatment.

The implantable implant is preferably made from a biocompatible material. The material may be bio-absorbable or non-absorbable. In some of these embodiments the material may be comprised of a bioabsorbable polymeric material. Suitable materials non-absorbable materials include but are not limited to polypropylene, nylon, biocompatible polymers, polymers of polyester, natural fiber, silk, or stainless steel wire or the like. Suitable bio-absorbable materials include, but are not limited to, plain or chromic catgut, polyglycolic acid (P.G.A.), polydioxanone (PDS), collagen, glycolide, bioabsorbable aliphatic polyesters, bioabsorbable polymers, or other similar materials.

Suitable biocompatible polymers include polyamides, polyolefins (e. g., polypropylene, polyethylene), nonabsorbable polyesters (e. g., polyethylene terephthalate), and bioabsorbable aliphatic polyesters (e. g., homopolymers and copolymers of lactic acid, glycolic acid, lactide, glycolide, para-dioxanone, trimethylene carbonate, s-caprolactone and blends thereof). Further, biocompatible polymers include film-forming bioabsorbable polymers; these include aliphatic polyesters, poly(amino acids), copoly(ether-esters), polyalkylenes oxalates, polyamides, poly(iminocarbonates), polyorthoesters, polyoxaesters including polyoxaesters containing amido groups, polyamidoesters, polyanhydrides, polyphosphazenes, biomolecules and blends thereof.

Biocompatible polymers further include film-forming biodurable polymers with relatively low chronic tissue response, such as polyurethanes, silicones, poly(meth)acrylates, polyesters, polyalkyl oxides (e. g., polyethylene oxide), polyvinyl alcohols, polyethylene glycols and polyvinyl pyrrolidone, as well as hydrogels, such as those formed from crosslinked polyvinyl pyrrolidinone and polyesters. Other polymers, of course, can also be used as the biocompatible polymer provided that they can be dissolved, cured or polymerized. Such polymers and copolymers include polyolefins, polyisobutylene and ethylene-a-olefin copolymers; acrylic polymers (including methacrylates) and copolymers; vinyl halide polymers and copolymers, such as polyvinyl chloride; polyvinyl ethers, such as polyvinyl methyl ether; polyvinylidene halides such as polyvinylidene fluoride and polyvinylidene chloride; polyacrylonitrile; polyvinyl ketones; polyvinyl aromatics such as polystyrene; polyvinyl esters such as polyvinyl acetate; copolymers of vinyl monomers with each other and with a-olefins, such as ethylene-methyl methacrylate copolymers and ethylene-vinyl acetate copolymers; acrylonitrile-styrene copolymers; ABS resins; polyamides, such as nylon 66 and polycaprolactam; alkyd resins; polycarbonates; polyoxymethylenes; polyimides; polyethers; epoxy resins; polyurethanes; rayon; rayon-triacetate; cellophane; cellulose and its derivatives such as cellulose acetate, cellulose acetate butyrate, cellulose nitrate, cellulose propionate and cellulose ethers (e. g., carboxymethyl cellulose and hydoxyalkyl celluloses); and mixtures thereof.

Implant 100 can be rigid or soft and/or pliable. In one embodiment (depicted by FIG. 1) the implant and its associated barbs are made from a single continuously formed piece of material. It is conceivable that in other embodiments the implant and the barbs can be formed from to separate pieces of material. In these embodiments the material may be the same kind of material or two entirely different materials. In one embodiment the implant may be comprised of a resiliently expandable elastomeric material that can recover its reduced size after insertion into the body. The elastomeric material may be comprised of a bioabsorbable polymeric material or other material having similar elastic properties. The implant may also have a hole, groove, clip, or other configuration at one or more ends for securing the implant to a needle or other tissue introducer during the implantation process. By using a tissue-introducing needle even a soft and/or pliable implant can be implanted deep within the tissue to secure the skin in place.

In some embodiments the material of implant 100 may provide fluid permeability through the material and may permit cellular ingrowth and proliferation into the interior of the material such as to allow it to bind into the surrounding tissues. The material may also be reticulated, such that it is comprised of an interconnected network of pores, either by being formed having a reticulated structure and/or undergoing a reticulation process.

The implantable device of the present invention can be suitable for long-term implantation and having sufficient porosity to encourage cellular ingrowth and proliferation, in vivo. Preferably, the implantable device is constructed such that it may be encapsulated and ingrown within the treatment area, and does not interfere with the function of the regrown cells and/or tissue, and has no tendency to migrate.

FIG. 2A depicts an implementation of a treatment area 200 that is modified with inserted implants 201. The treatment area comprises a subdermal area 202 in which adipocyte expansion has stretched the fibrous septae 203 and caused a portion 204 of the skin surface 206 to be held down while other sections 205 bulge outward, resulting in cellulite. The implants are inserted or injected percutaneously into the subdermal fat layer, or in the layer between the subdermal fat layers and the skin. In some embodiments, the implants are inserted at non-parallel angles. In other embodiments (depicted by FIGS. 4A and 4D), the skin 206 is first tightened by stretching or shifting the surface of the skin 206 to disoreint the fibrous structures 203 beneath. Implants 201 may then be inserted to retain the skin in a stretched or shifted configuration, thus realigning the fibrous structures in a new direction, in most cases non-perpendicular to the surface of the skin.

It is the intent of the invention that, after implantation, the healing process will develop new scar tissue where the implant was implanted, forming new fibrous structures and other connective tissue. As illustrated by FIG. 2B, where the structures were originally oriented in a parallel fashion (and perpendicular to the skin), the new scar tissue or connective tissue growth 301 will be in a much more random orientation consistent with the orientation of the implants. Eventually this connective tissue will contract and harden, holding the skin at a non-flexible length; however, the chambers 302 between the septae will now be smaller, resulting in a lesser ability to retain excess adipocyte. In some embodiments, the period of implantation will be at least sufficient for cellular ingrowth and proliferation to commence, for example, in at least about 4-8 weeks. In other embodiments, the material of implants 201, 100 may be broken down in tissue after a given period of time, which depending on the material, may be from ten days to eight weeks.

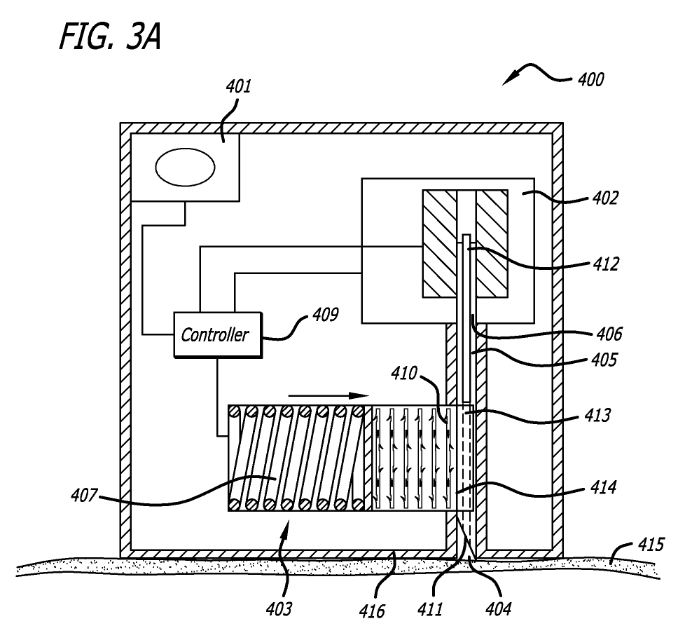

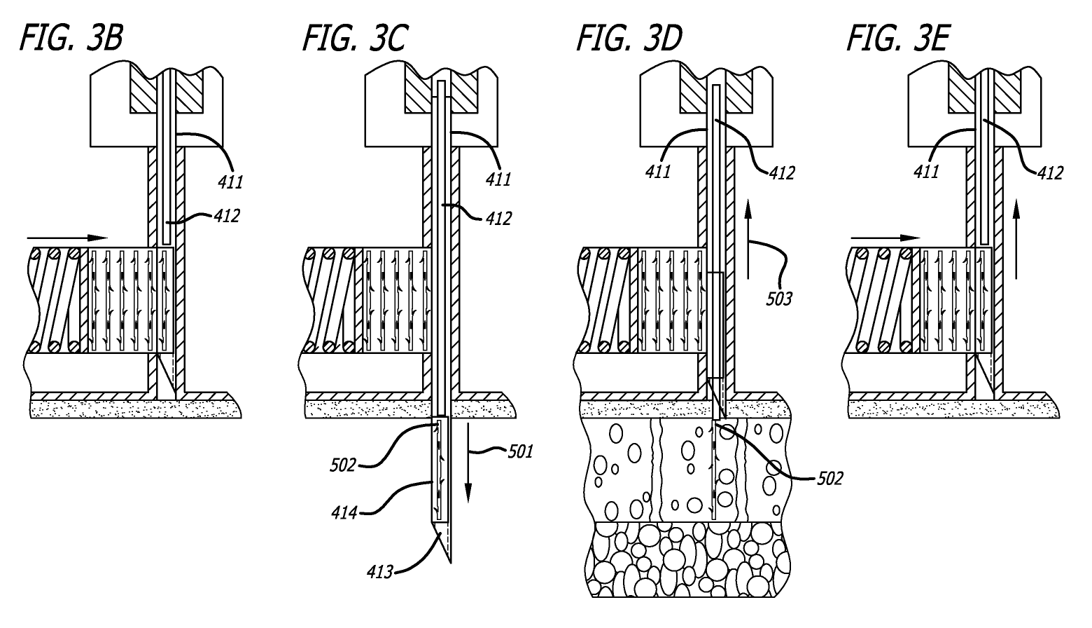

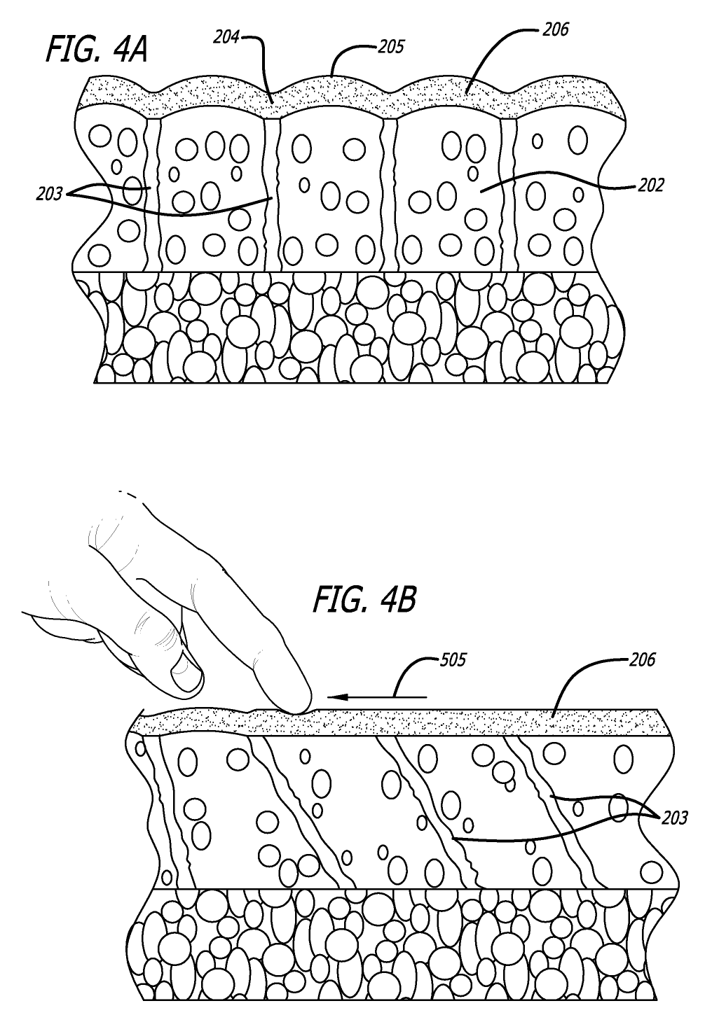

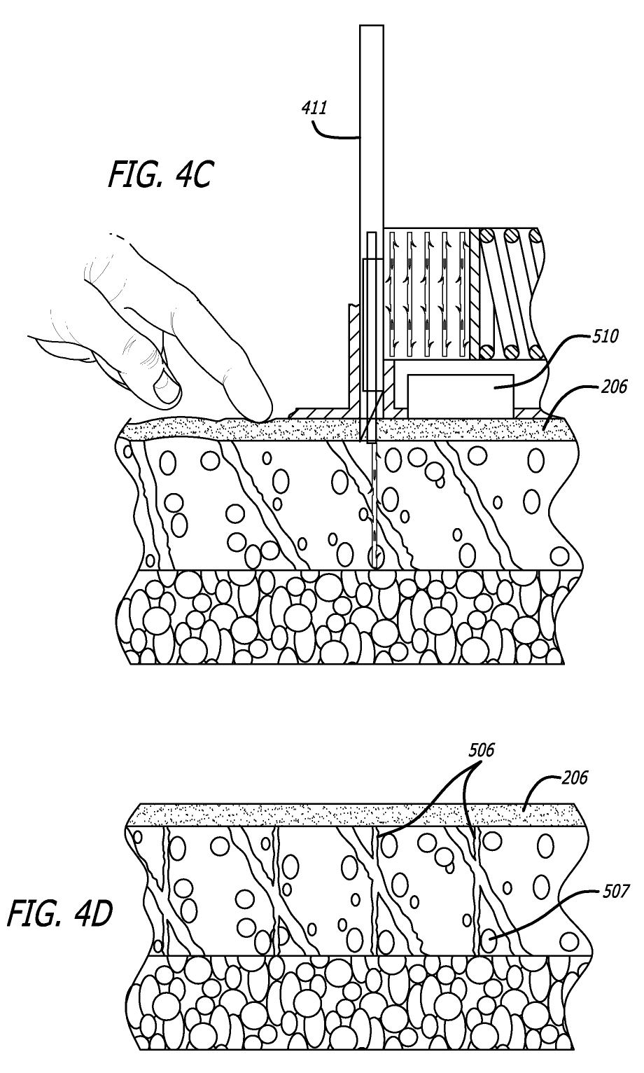

FIG. 3A depicts a device for implementing the method of the present invention. Device 400 is used to inject a large number of implants in order to treat a large area. The device includes a control mechanism 401, a controller 409 an implant actuator 402, an implant loader 403, and an implant ejection port 404. An introducing channel structure 405 extends downwardly from the lower end 406 of the implant actuator to the ejection port 404. Channel structure 405 is preferably situated adjacent the adjacent to loader 403 such that an implant 410 may be received from the loader. In some embodiments loader 403 will be a non-removable part of device 400. In other embodiments loader 403 may be configured in the form of a magazine cartridge (not illustrated) which is inserted onto or into a loader receptacle. Loader 403 may hold one or more or a collection of individual implanted sutures/implants 410, which are individually implanted with each actuation of implant actuator 402. Where more than one implant 410 may be loaded in loader 403, the loader may have a push cartridge or rod 407 for introducing implants 410 into the introducing channel. Implants 410 are kept aligned, and by virtue of the push rod 407, pushed one-by-one into the introducing channel 405 in pace with the firings from the implant actuator 402. According to one embodiment, when the push-rod 407 of loader 403 has reached its extreme position, furthest forward in the loader (when the last implant has been loaded), there being no implant in the guide, loader 403 will signal controller 409 to cease further firing of the implant actuator 402.

Implant actuator 402 preferably comprises an introducing needle 411 and a piston 412. Needle 411 is configured to move within introducing channel 405 and, when in a deployed position, a distal end of the needle extending out ejection port 404 and into a tissue surface 415. Introducing needle 411 preferably has a narrow or slim profile and a sharp distal end (e.g., a needle) for minimally invasive insertion into a tissue surface. Needle 411 is typically hollow or has a groove 413, and, in the illustrated embodiment-introducing needle 411 may have a lateral recess 414, configured for receiving an implant from the loader. When moved into position, an implant 410 in the forward firing position of the loader is then received by the introducing needle for insertion of the implant percutaneously into the treatment area.

Implant actuator 402 may also comprise a piston 412 for providing a driving force 501 to an implant positioned in the introducing needle 411 to force it out of ejection port 404 and into a tissue 415. The piston is preferably configured to slide within hollow portion or groove 413 within the needle. Groove 413 preferably passes through lateral recess 414. Piston 412 and needle 411 are configured such that when piston 412 extends into the lateral groove 413 it will push an implant within the recess 414 through groove 413 of introducing needle 411 toward the end of the needle. In such a case, piston 412 applies force 501 to introducing needle 411 and a proximal end 502 of an implant. Turning to FIGS. 3A-3E the device is configured to inject an implant into tissue 415 on each actuation of implant actuator 402. As illustrated by FIG. 3C, introducing needle 411 is preferably associated with piston 412 such that when the piston is fired at least a portion of a distal end of introducing needle 411 extends out of ejection port 404. After firing the piston and introducing needle 411 is in the tissue, introducing needle 411 is retracted back into the device while piston 412 remains, at least temporarily in place. As depicted by FIG. 3D, when introducing needle 411 is retracted, and by virtue of piston 412 remaining in place, piston 412 applies a force to a proximal portion 502 of the implant forcing it out of introducing needle 411. The force applied to proximal portion 502 is inversely proportional to the force 503 of the needle retraction. As shown by FIG. 3E, once introducing needle 411 has retracted to a certain point piston 412 follows by retracting with needle 411 to its original position. Returning introducing needle 411 to its original position triggers loader 403 to load another implant 410 into implant recess 414 of introducing needle 411.

In those embodiments where the implant may be more rigid by nature, the implant may be received into lateral recess 414. As introducing needle 411 is inserted into the skin the implant is ejected from the recess either by the ejection mechanism described or by the friction force of the barbs 101 located on the implant. In other embodiments, in which the implant material may be more pliable, the introducing needle may be similar to a sewing needle. The introducing needle threads the forward most implant as the needle passes through the positioning guide. The implant 410 is then inserted in a distal direction into the tissue, maintains traction in the tissue via its barbed structure, the introducing needle unthreading the implant as the needle retracts for the next firing.

The implant actuator of the implant injection device in accordance with this invention is preferably electrically powered, however, may be pneumatically powered (e.g. by compressed air or CO₂ cartridge), or powered by any other mean known in the art. In certain embodiments (e.g. sewing needle configuration) the piston may be eliminated and/or replaced by the introducing needle. The depth of the needle, the depth of the piston, and the speed of deployment of the device are all adjustable and controlled by a controller 401 on the device which controls the needle and piston actuators. The implant actuator may be configured for rapid-fire activation of the introducing needle. The pace of injection/retraction set by controller 401 may be preselected using a control located on the device. For example, the device may either be used in single shot mode, where actuation of the power button generates a single implant or it may be in multi-shot mode where holding the button continually implants sutures until it is released.

In certain embodiments controller 409 may also control the angle of penetration. The device preferably comprises a tissue apposition surface 416 that is typically positioned perpendicular to the surface of the skin. In some embodiments the positioning guide 405 and introducing needle 411 therein is initially positioned perpendicular to the skin. The device injects the implants percutaneously into the subdermal fat layer, or in the layer between the subdermal fat layers and the skin as depicted by FIG. 4B. The skin may be first tightened by stretching or shifting the surface of the skin to disoreint the fibrous structures 203 beneath. Implants 100 may then be injected to retain the skin in a stretched or shifted configuration (shown by FIG. 4C), thus realigning fibrous structures 203 in a new direction 505, in most cases non-perpendicular to the surface of skin 206. In other embodiments, guide 405 and introducing needle 411 may be positioned at an angle relative to the skin so that the implants can be injected at non-parallel angles. As illustrated by FIG. 4D, new scar tissue will develop where implant 100 was implanted, forming new fibrous structures and other connective tissue 506, thereby creating smaller chambers 507 between the septae and resulting in the tissue having a decreased ability to retain excess adipocyte. The angle of penetration may be selected by operating the control, or, in some embodiments, by manual configuration of the guide or positioning of the device itself.

It is preferable that the needle, piston, sutures, and implant loader are all sterile components. The piston may be reusable and can be made of a material that can be autoclaved, ETO, gamma sterilized, or other suitable material. It is also preferable that the implant loader, implants, and implant actuator are disposable. The device may be further configured to incorporate insulation liner within the positioning guide to keep the implant loader, implants, and implant actuator in fluid isolation from the other parts of the device.

In some embodiments (shown by example in FIG. 4C) in the device further comprises an energy device 510. In accordance with these embodiments, energy device 510 may be configured to apply energy to the tissue after the implants has been inserted into the treatment area. In some embodiments the energy may also be applied to shrink the implants after they have been deployed under the skin. In these embodiments if the skin was shifted or stretched prior to implantation the implant may be heated to further tighten and/or further secure the area of implantation and/or to shrink the implants and create a tightening of the subcutaneous tissues. In some embodiments the energy may be used to create damage sites along the implants that will heal as fibrous structures. The thermal energy device may include a microwave, conductive heat, ultrasound, or RF.

The device operates in four states:
(1) A single suture is loaded into the needle, while the needle and piston are retracted. (FIG. 3B).
(2) The needle and piston are concurrently deployed to a fixed depth that is set in the device. (FIG. 3C).
(3) The needle is retracted while the piston remains in place, thus depositing the suture in the tissue at a predetermined depth. (FIG. 3D).
(4) The piston is retracted. (FIG. 3E).

One method of using the present invention is directed to providing an implant injection device configured to subcutaneously implant implants percutaneously into the subdermal fat layer, or in the layer between the subdermal fat layers and the skin.

(1) A device is provided having a control, an implant actuator, an implant loader, an introducing channel and an ejection port. The loader may contain at least one implant, but may also comprise cartridge full of implants.

(2) The device is configured by the operation of a control on the device. The depth of the needle, the depth of the piston, angle of penetration, and the speed of deployment of the device may all be adjusted and/or controlled through control mechanism 401 and controller 409.

(3) Preparing the treatment area comprising a section of skin and/or underlying subdermal layers is configured for treatment. In some embodiments the skin may be prepared by applying a treatment solution to the skin. The solution may be applied to the surface, or, in certain embodiments, the treatment solution can be injected. The treatment solution may comprise a local anesthetic or pain relieving solution and/or an antibiotic, or a combination of treatment solutions useful in similar medical procedures. In some embodiments preparing the treatment area may comprise stretching or skewing the skin from its original configuration, thus temporarily realigning the fibrous structures in a new direction, and in most cases a non-perpendicular to the surface of the skin. (Shown by FIG. 4B).

(4) The ejection port of the device is positioned proximal to the treatment area. This may entail pressing the tissue apposition surface against a tissue having dimpled cellulite. In some embodiments this entails applying pressure on a hand piece. In other embodiments this entails using a vacuum enabled handpiece to bring the tissue into contact with the tissue apposition surface.

(5) A control is actuated to fire an implant which has been preloaded onto, or within, the introducing needle is injected to the treatment area at the desired angle. If the skin has been stretched or shifted then the implants may be injected to retain the skin in the skewed configuration, thus realigning the fibrous structures in a new direction, in most cases non-perpendicular to the surface of the skin.

(6) The previous step is repeated as many times as needed for proper treatment of the treatment area, to reduce the appearance of cellulite, and/or to maintain the skin in its new position.

(7) The device is removed from the treatment area and/or moved to a new treatment area. The device may be moved substantially parallel to the treatment area while the control is actuated so that multiple implants can be fired into the treatment area at more than one location.

(8) Once the implants are anchored within the tissue, a thermal energy such as microwave, conductive heat, ultrasound, RF may be applied to the tissue. In one embodiment the energy may be used to create damage sites along the mesh that will heal as fibrous structures. In another embodiment the energy may be used to shrink the implants when in place in the subdermal fat and create a tightening of the subcutaneous tissues.

The forgoing description for the preferred embodiments of the invention has been presented for the purposes of illustration and description. It is not intended to be exhaustive or to limit the invention to the precise form disclosed. For example, not all of the steps illustrated, or precise sequence, may be required to perform the method of the invention. More steps may be included consistent with the embodiments herein and the sequence may be varied consistent with these embodiments. Indeed, many modifications and variations are possible in light of the above teaching. It is intended that the scope of the invention not be limited by this detailed description, but by the claims and the equivalents to the claims appended hereto.

Although the present invention has been described in detail with regard to the preferred embodiments and drawings thereof, it should be apparent to those of ordinary skill in the art that various adaptations and modifications of the present invention may be accomplished without departing from the spirit and the scope of the invention. Accordingly, it is to be understood that the detailed description and the accompanying drawings as set forth hereinabove are not intended to limit the breadth of the present invention.

We claim:

1. An implant injection device, comprising:
a control;
an implant actuator;
an implant loader, wherein the loader holds at least one implant;
ejection port; and
a positioning guide disposed between the implant actuator and the ejection port passing adjacent to the feed magazine,
wherein the implants comprise a biocompatible material having a nominal length from about 5 mm to 20 mm, the length being substantially longer than a nominal outer diameter of the implant,
wherein the device is configured to inject at least one implant into a treatment area comprising the subdermal fat layer, or the layer between the subdermal fat layers and the skin, when the control is actuated to create a new fibrous structure in the treatment area to reduce the appearance of the cellulite.

2. The implant injection device of claim 1, wherein the implant has at least two barbs preferably configured in an opposing pattern and mirrored along each respective half of the implant.

3. The implant injection device of claim 1, wherein the implant actuator is configured to push the implant percutaneously into the treatment area.

4. The implant injection device of claim 1, further comprising:
an introducing needle,
wherein the introducing needle is configured to receive an implant into a recessed portion of the introducing needle, and to percutaneously insert the implant into the treatment area.

5. The implant injection device of claim 1, further comprising:
an introducing needle,
wherein the introducing needle is configured to thread the implant as it is forwarded to the introducing needle by the implant loader, and to push the implant percutaneously into the treatment area.

6. The implant injection device of claim 1, wherein the control manipulates the depth of the needle, the speed of deployment, or angle of penetration.

7. The implant injection device of claim 1, wherein the device is electrically powered.

8. The implant injection device of claim 1, further comprising:
a thermal energy device associated with the tissue apposition surface and configured to apply a thermal energy to the treatment area.

9. A method of injecting surgical implants for treating cellulite, comprising:
providing an implant injection device, comprising, a control, an implant loader, an implant ejection port;
preparing a treatment area comprising a section of skin;
inserting an implant into the implant loader, the implant comprising a biocompatible material having a nominal length from about 5 mm to 20 mm, the length being substantially longer than a nominal outer diameter of the implant, wherein the implant has at least two barbs preferably configured in an opposing pattern and mirrored along each respective half of the implant;
positioning the implant ejection port proximal the treatment area; and
actuating the control to inject at least one implant into the treatment area to create a new fibrous structure in a subdermal tissue to reduce the appearance of the cellulite.

10. The method of claim 9, wherein the implant injection device is moved substantially parallel to the treatment area while the control is actuated, and wherein at least two implants are fired into the treatment area in at least two locations.

11. The method of claim 9, further comprising:
manipulating a control to set a speed of deployment.

12. The method of claim 9, further comprising:
manipulating a control to set a speed of deployment.

13. The method of claim 9, further comprising:
applying a thermal energy to the treatment area.

14. The method of claim 9, wherein preparing the treatment area comprises:
stretching at least a portion of the skin.

15. The method of claim 14, wherein injecting the implant supports the at least a portion of the skin in a new position.

16. A surgical implant, comprising:
a biocompatible material having a nominal length from about 5 mm to 20 mm, the length being substantially longer than a nominal outer diameter of the implant,
wherein the implant has at least two barbs preferably configured in an opposing pattern and mirrored along each respective half of the implant.

17. The surgical implant of claim 16, wherein the biocompatible material has a nominal outer diameter from about 0.25 mm to 0.50 mm.

18. The surgical implant of claim 16, wherein the biocompatible material is rigid.

19. The surgical implant of claim 16, wherein the biocompatible material is pliable.

20. The surgical implant of claim 16, wherein a cross-section of the implant is substantially flat.

21. The surgical implant of claim 16, wherein a cross-section of the implant is substantially round.

22. The surgical implant of claim 16, wherein the biocompatible material is bioabsorbable.

23. The surgical implant of claim 16, wherein the biocompatible material is a resiliently expandable elastomeric material that can recover its reduced size after insertion into the body.

24. The surgical implant of claim 16, wherein the biocompatible material of the implant provides fluid permeability through the material and permits cellular ingrowth and proliferation into the interior of the material such as to allow it to bind into the surrounding tissues.

25. The surgical implant of claim 16, wherein the biocompatible material is reticulated, such that it is comprised of an interconnected network of pores, either by being formed having a reticulated structure and/or undergoing a reticulation process.
